(19) United States
(12) Patent Application Publication
(10) Pub. No.: US 2007/0100223 A1
(43) Pub. Date: May 3, 2007

(54) Method and system for cardiac imaging and catheter guidance for radio frequency (RF) ablation

(76) Inventors: Rui Liao, Plainsboro, NJ (US); Frank Sauer, Princeton, NJ (US); Yiyong Sun, Lawrenceville, NJ (US); Chenyang Xu, Allentown, NJ (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/545,383

(22) Filed: Oct. 10, 2006

Related U.S. Application Data
(60) Provisional application No. 60/726,597, filed on Oct. 14, 2005.

(51) Int. Cl. A61B 5/05

(52) U.S. Cl. 600/407

(57) ABSTRACT

A method for imaging for cardiac catheter guidance comprises displaying a two-dimensional (2D) image of a heart, including a catheter; registering and blending the 2D image and a three-dimensional (3D) image of the heart to derive a blended image; displaying the blended image and the 3D image; and extracting an image of the catheter and inserting it into the 3D image.

### CROSS-REFERENCE TO RELATED APPLICATION AND CLAIM OF PRIORITY

Specific reference is hereby made to copending U.S. Provisional Patent Application No. 60/726,597 (Attorney Docket No. 2005P18854US) filed Oct. 14, 2005, in the names of inventors Rui Liao, Chenyang Xu, Yiyong Sun, and Frank Sauer, and entitled Method and System for Catheter RF ablation Using 2D- 3D Registration, and whereof the disclosure is hereby incorporated herein by reference and whereof the benefit of priority is claimed.

### FIELD OF THE INVENTION

The present invention relates generally to computerized imaging as may be utilized for locating particular points in an organ and, more particularly with providing an image of a heart and catheter for facilitating placement and manipulation of the catheter for treating atrial fibrillation.

### BACKGROUND OF THE INVENTION

Atrial fibrillation (AFIB) is a leading cause of stroke in human beings and one of the most common heart rhythm disorders. Typically, it involves the occurrence of extra firing of cells in the heart. At the present time, treatments for atrial fibrillation typically include the use of anti-arrhythmic drugs, cardiac surgery, using an external defibrillator, or by radio-frequency (RF) catheter ablation of sites pertaining to the excess firing of cells. Depending on various factors, including the condition of the patient, a selection is made for the most suitable course of treatment. RF ablation in particular has the potential of becoming a therapy of choice, as an alternative to other methods for treating atrial fibrillation.

In order to guide the process of finding the site of origin where the excess firing of cells occur, modem cardiac mapping systems have made it possible to draw the heart as a three-dimensional (3D) model and can provide real-time electrical activation information. Such mapping systems include CARTO XP™, EnSite 3000™ and Constellation™, all of which require special catheters that are much more expensive than normal catheters, in order to provide the position of the pacing catheter accurately. See for example, Savard, P., Sierra, G., LeBlanc, A., Leonard, M., Nadeau, R., “Prototype of a Fluoroscopic Navigation System to Guide Catheter Ablation in Cardiac Arrhythmias, First Experiences”, Proceedings of XXV Conference of The IEEE Engineering in Medicine and Biology Society 2003, 138-142.

More recently, registration of high-resolution 3D atrial computerized tomography (CT) and magnetic resonance (MR) volumes with cardiac mapping systems provides a more realistic picture of patients' heart anatomy and electrical activities, thereby representing a major technological advance in diagnosing complex arrhythmias. See, for example, Sun, Y., Azar, F., Xu, C., Hayam, G., Preiss, A., Rahn, N., Sauer, F., “Registration of High Resolution 3D Atrial Images with Electroanatomical Cardiac Mapping: Evaluation of registration methodology”, SPIE 2005, whereof the disclosure is hereby incorporated herein by reference to the extent it is not incompatible with the present invention.

Newly released software described in the afore-mentioned publication by Sun, Y. et al., nevertheless requires expensive cardiac mapping systems and specialized catheters to provide the 3D position of the catheter for 3D/3D registration and catheter tracking. A 2D/3D intensity-based registration algorithm specialized for the application of radiation therapy is introduced in Wein, W., “Intensity Based Rigid 2D-3D Registration Algorithms for Radiation Therapy”, Master thesis, 2003; registration and other topics are reviewed in this work, whereof the disclosure is hereby incorporated herein by reference to the extent it is not incompatible with the present invention.

### SUMMARY OF THE INVENTION

The need is herein recognized for a more cost-effective navigation system for the human heart than is provided by existing cardiac mapping systems, without seriously sacrificing their associated 3D visualization capability, in order to meet the demands of an aging population of patients who are more likely to experience severe arrhythmias and whose medical expenses are soaring, even for those who have insurance coverage. The present invention is generally applicable to images obtained by MR and CT imaging systems. Accordingly, the following description sometimes refers to one or the other or both types of images; this should not be construed to limit the description to either. It is also anticipated that the invention will also be useful in conjunction with other analogous imaging systems. Typically, a reference to an image may also be construed to mean an actual image or an equivalent image dataset.

In accordance with an aspect of the invention, a method for imaging for cardiac catheter guidance comprises displaying a two-dimensional (2D) image of a heart, including a catheter; registering and blending the 2D image and a three-dimensional (3D) image of the heart to derive a blended image; displaying the blended image and the 3D image; and extracting an image of the catheter and inserting it into at least one of the blended image and the 3D image.

In accordance with another aspect of the invention, a method for imaging for cardiac catheter guidance, comprises: displaying a two-dimensional (2D) image of a heart; registering the 2D image and a three-dimensional (3D) image of the heart; deriving a blended image by fusion of the 2D image and the 3D image; displaying the blended image; extracting a given feature image from the 2D image; and inserting the feature image into the 3D image.

In accordance with another aspect of the invention, steps of the method are performed under automatic control.

In accordance with another aspect of the invention, the step of deriving a blended image by fusion comprises blending of the 2D image and the 3D image.

In accordance with another aspect of the invention, the step of blending comprises superimposing one of the 3D image and the 2D image over the other.

In accordance with another aspect of the invention, relative weights of the superimposed 3D image and the 2D image are selectable by operator control.

In accordance with another aspect of the invention, the registering comprises utilizing intensity-based registration.

In accordance with another aspect of the invention, the registering comprises utilizing feature-based registration.

In accordance with another aspect of the invention, the step of extracting a given feature image comprises extracting a catheter image.

In accordance with another aspect of the invention, a method for imaging for cardiac catheter guidance, comprises: acquiring a two-dimensional (2D) image of a heart by fluoroscopy; acquiring a three-dimensional (3D) image of the heart by at least one of (a) computerized tomography (CT) imaging and (b) magnetic resonance (MR) imaging; registering the 2D and the 3D images; generating a blended image from the 2D and the 3D images; extracting an image of a catheter from the 2D image; displaying the blended image; and inserting the image of the catheter into at least one of the blended image and the 3D image.

In accordance with another aspect of the invention steps of the method are performed under automatic control.

In accordance with another aspect of the invention a method in accordance with the invention, comprises optionally displaying the 3D image.

In accordance with another aspect of the invention the step of generating the blended image comprises: superimposing the 2D image on top of the 3D image.

In accordance with another aspect of the invention, relative intensities of the 2D image and the 3D image are controllable by a user.

In accordance with another aspect of the invention the step of displaying the blended image and the 3D image comprises: juxtaposing the 3D image and the blended image.

In accordance with another aspect of the invention the step of displaying the blended image and the 3D image comprises: superimposing a further image on the blended image.

For example, this could comprise a surface extraction from the volume image.

In accordance with another aspect of the invention the step of registering comprises utilizing intensity-based registration.

In accordance with another aspect of the invention the step of utilizing intensity-based registration comprises: digitally reconstructing a radiography (DRR) image from volumetric data from the 3D image; and comparing quantitatively the DRR image with the 2D image to derive a rigid transformation relating an isocenter coordinate of the fluoroscopy to that of the 3D image.

In accordance with another aspect of the invention the step of utilizing intensity-based registration comprises: utilizing 2D images from a plurality of views for intensity-based registration so as to increase registration accuracy.

In accordance with another aspect of the invention the step of utilizing intensity-based registration comprises: utilizing 2D images from a plurality of views for intensity-based registration.

In accordance with another aspect of the invention, the step of utilizing intensity-based registration comprises: utilizing 2D images from a plurality of views for intensity-based registration so as to increase registration accuracy with respect to depth estimation.

In accordance with another aspect of the invention, steps are performed under automatic control.

In accordance with another aspect of the invention, the step of utilizing intensity-based registration comprises injecting contrast agent to highlight vessels to improve registration.

In accordance with another aspect of the invention, wherein the step of registering comprises utilizing feature-based registration.

In accordance with another aspect of the invention, the step of utilizing feature-based registration wherein: the step of acquiring a 2D image of a heart by fluoroscopy comprises utilizing a C-arm mounting for the fluoroscopy; selecting landmarks corresponding to respective physical features present in a plurality of 2D images captured from different views corresponding respectively to different respective parameter settings of the C-arm; computing true 3D positions of the physical features by using the parameter settings; identifying landmark points corresponding in volumetric data of the 3D image; and aligning the true 3D positions with corresponding respective landmark points for achieving registration.

In accordance with another aspect of the invention the step of selecting landmarks comprises utilizing the parameter settings including any of angulations, zooming effects, table movements, and similar parameter changes.

In accordance with another aspect of the invention the landmark points comprise at least - 3 pairs of points.

In accordance with another aspect of the invention the landmark points comprise at least one pair of points.

In accordance with another aspect of the invention, registering and fusing the 2D and the 3D images for generating a blended image from the 2D and the 3D images.

In accordance with another aspect of the invention, a method, comprises adding a color component to the 3D image.

In accordance with another aspect of the invention, a method comprises extracting and highlighting edges in the 3D image.

In accordance with another aspect of the invention, a method comprises highlighting the catheter image shown in the 2D image by any of background suppression, edge enhancement filtering, and automatic window-leveling.

In accordance with another aspect of the invention a method for imaging for cardiac catheter guidance, comprises: acquiring a two-dimensional (2D) image of a heart by fluoroscopy including a catheter image; acquiring a dataset for a three-dimensional (3D) image of a heart; registering the 2D and the 3D images; generating a blended image from the 2D and the 3D images after the registering; extracting an image of the catheter; displaying the blended image and the 3D image; and inserting the catheter image into at least one of the blended image and the 3D image.

In accordance with another aspect of the invention a method for imaging for cardiac catheter guidance, comprises: displaying a two-dimensional (2D) image of a heart, including a catheter image; extracting an image of the catheter; deriving a three- dimensional (3D) image of the heart; registering the 2D and 3D images; blending the 2D and 3D images to derive a blended image; displaying the blended image and the 3D image; and inserting the catheter image into the blended image.

In accordance with another aspect of the invention, a system for imaging for cardiac catheter guidance, comprises: a memory device for storing a program and other data; and a processor in communication with the memory device, the processor being operative with the program to perform: acquiring a two-dimensional (2D) image of a heart by fluoroscopy including a catheter; acquiring a three-dimensional (3D) image of the heart by at least one of (a) computerized tomography (CT) imaging and (b) magnetic resonance (MR) imaging; registering the 2D and the 3D images; generating a blended image from the 2D and the 3D images after the registering; extracting an image of the catheter; displaying the blended image and the 3D image in combination; and inserting the image of the catheter into at least one of the blended image and the 3D image.

In accordance with another aspect of the invention, a computer program product comprises a computer useable medium having computer program logic recorded thereon for program code for performing imaging for cardiac catheter guidance, by: acquiring a two-dimensional (2D) image of a heart by fluoroscopy including a catheter; acquiring a three-dimensional (3D) image of the heart by at least one of (a) computerized tomography (CT) imaging and (b) magnetic resonance (MR) imaging; registering the 2D and the 3D images; generating a blended image from the 2D and the 3D images; extracting an image of the catheter; displaying the blended image and the 3D image in combination; and inserting the image of the catheter into at least one of the blended image and the 3D image.

In accordance with another aspect of the invention, system for imaging for cardiac catheter guidance, comprises: memory apparatus for storing a program and other data; and processor apparatus in communication with the memory apparatus, the processor apparatus being operative with the program to perform: acquiring a two-dimensional (2D) image of a heart by fluoroscopy including a catheter; acquiring a three-dimensional (3D) image of the heart by at least one of (a) computerized tomography (CT) imaging and (b) magnetic resonance (MR) imaging; registering the 2D and the 3D images; generating a blended image from the 2D and the 3D images after the registering; extracting an image of the catheter; displaying the blended image and the 3D image in combination; and inserting the image of the catheter into at least one of the blended image and the 3D image.

### BRIEF DESCRIPTION OF THE SEVERAL VIEWS OF THE DRAWINGS

The invention will be more fully understood from the detailed description which follows, in conjunction with the drawings, in which:

FIG. 1 shows a schematic representation of processing steps in accordance with principles of the present invention;

FIG. 2 shows a schematic representation of processing steps in accordance with principles of the present invention, including representative images for various steps;

FIG. 3 shows experimental results in accordance with principles of the present invention;

FIG. 4 shows catheter image enhancement in accordance with principles of the present invention; and

FIG. 5 shows in schematic form the application of a programmable digital computer for implementation of the invention.

### DETAILED DESCRIPTION OF THE INVENTION

The present invention is concerned with registration and fusion of 3D atrial CT and MR volumes with two-dimensional (2D) fluoroscopic images through automatic 2D/3D registration techniques, involving alignment between a volumetric data set with data of a projected image. Generally, registration is the bringing into spatial alignment of image data originating from different devices and/or that has been obtained at different times. The augmented visualization of highly detailed 3D data can help physicians move the catheter with greater confidence and guide it more precisely to specific areas of the heart responsible for generating the arrhythmia, thereby improving ablation success rates and lowering risks related to the procedure.

Since fluoroscopy is already used by radiologists on a routine basis to guide RF ablation, and an expensive cardiac mapping system together with a specialized catheter are no longer required, the navigation system in accordance with the present invention will help to reduce medical costs and increase the number of ablation procedures that can be carried out in a given facility on a yearly basis. This is especially important for medium-sized community hospitals, for example, that cannot afford to purchase a single conventional mapping system, typically costing in the neighborhood of $400,000.

In accordance with principles of the present invention, automatic 2D/3D registration techniques are applied to fuse high-resolution 3D CT and MR volumes with 2D fluoroscopy for the clinical application of atrial fibrillation (AFIB) catheter ablation. See the afore-mentioned thesis by Wein, W.

It has been demonstrated in the afore-mentioned publication by Sun, Y. et al. that the display of a detailed anatomic 3D volume during catheter ablation enables physicians for the first time to track the movement of the catheter within an exact representation of the patient's heart, allowing for more precise navigation of catheters to targeted points within the heart. The present invention provides a cost-effective alternative to the 3D-3D fusion disclosed in the afore-mentioned publication by Sun, Y. et al. by eliminating the requirement for expensive cardiac mapping systems and specialized catheters. Rather, the 3D volume is registered through 2D/3D registration techniques and fused using a special blending effect with the 2D projected fluoroscopic image to guide the procedure of catheter ablation.

While the foregoing is, in a sense, not a true 3D navigation process because the 3D location of the catheter is not identified, the image of the 3D volume obtained through special rendering techniques reveals important 3D information that can greatly assist physicians in visualizing the location of the invasive medical instruments utilized and in minimizing the number of incisions and trials needed. The system in accordance with the present invention can be regarded as a “2.5-D” navigation system for cost-effective catheter ablation.

The invention will be further described below by way of exemplary embodiments illustrating the best mode known to the present inventors of practicing the invention, in conjunction with the figures.

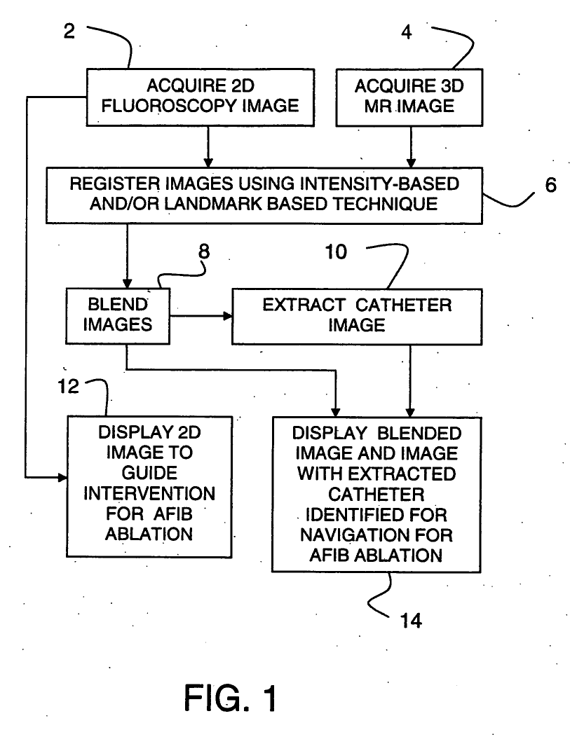

Three principal components are utilized in an embodiment of the present invention to be described next. FIG. 1 shows a schematic block diagram illustrating a method in accordance with principles of the present invention for providing a conventional 2D navigation system for AFIB in conjunction with a quasi 3D navigation system for AFIB, utilizing 2D/3D registration, and herein referred to as a 2.5D system, as mentioned above.

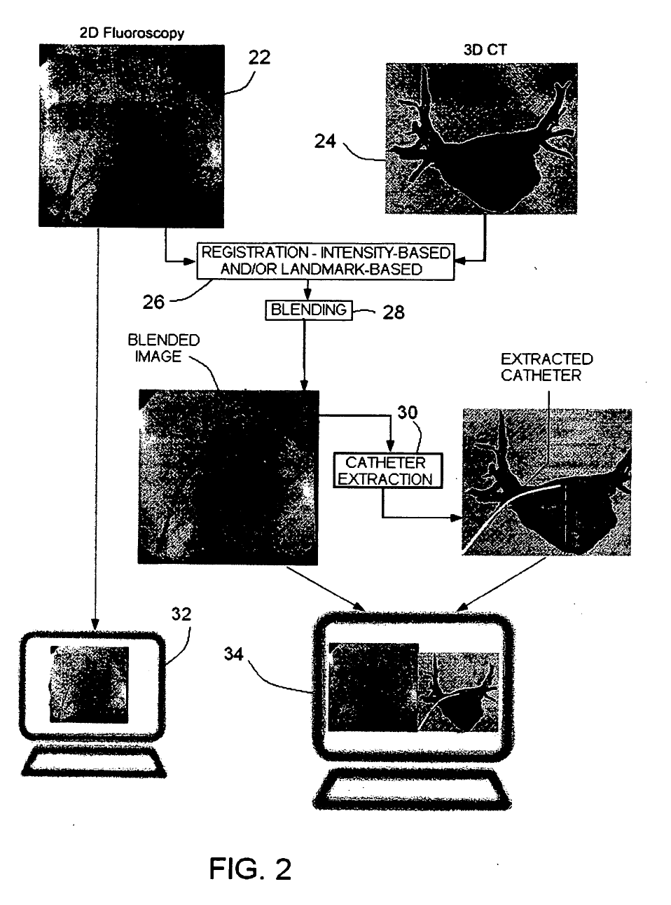

FIG. 2 shows the schematic of FIG. 1 wherein images are also shown to further illustrate the various stages and steps in accordance with the present invention. Components in FIG. 2 generally corresponding to components in FIG. 1 are indicated by the same reference numeral augmented by 20.

The first component relates to data acquisition, as indicated by boxes 2 and 4 in FIG. 1, and 22 and 24 in FIG. 2. A 3D volume, e.g. a 3D CT and/or MR volume is typically acquired pre-operatively. A 3D angio volume can be acquired during the operation through DynaCT™. A 2D fluoroscopic image is acquired continuously in the operating room to monitor the surgical procedures and guide the surgery.

A second component relates to 2D/3D registration, as indicated at 6 in FIG. 1. For one applicable technique, using intensity-based registration, digitally reconstructed radiography (DRR) is computed from volumetric data and is compared quantitatively with the fluoroscopic image in order to determine the rigid transformation relating the isocenter coordinate of the 2D fluoroscopy to that of the pre-operative CT and/or MR. Fluoroscopic images from multiple views can be used to improve the registration accuracy, especially in depth estimation. Common structures in both 3D volume and 2D fluoroscopy, especially bony structures such as ribs and spine, are the key features that drive the intensity-based registration. Contrast agent can also be injected to highlight the vessels that are likely to be helpful for registration.

For a second applicable technique, using feature-based registration, landmarks corresponding to the same physical points, such as salient points on pulmonary veins (PV), can be picked on the fluoroscopic images that are captured from different views. The true 3D positions of the physical points picked can then be computed using the parameter settings of the C-Arm for the different views such as angulations, zooming effects, table movements, and so forth. Registration is achieved by aligning the calculated 3D points with the corresponding real 3D points picked on the 3D volumetric data. At least one pair of points is needed to achieve the estimation in translation, and at least three pairs of points are needed if six-parameter rigid-body transformation is required.

The technique using intensity-based registration typically requires less operator involvement and interaction than the technique using feature-based registration and is therefore more adaptable to automatic operation.

A third component relates to 2D/3D Image Fusion. In a broader sense, this represents the fusion of information from a plurality of source images. In the present embodiment, 3D volumetric data is rendered using a volume rendering technique (VRT) and is superimposed on top of 2D fluoroscopy.

Colors can be added to the rendered VRT image for colored display, and edges of the rendered VRT image can be extracted and highlighted. 2D Fluoroscopy: catheters shown in 2D fluoroscopy can be highlighted through background suppression, edge enhancement filtering and automatic window-leveling for enhanced display.

Blending, which in the present embodiment may be considered as part of an overall fusion process is indicated by step 8 in FIG. 1. Blending is obtained by a user fading between the rendered VRT image and 2D fluoroscopy by changing the blending value through a graphical user interface (GUI). Blending may also involve catheter image extraction (10). The blended image is displayed by a display system at 14 and the 2D fluoroscopic image is displayed by a display unit at 12.

Both the blended image and the 2D fluoroscopic image are available for display at 12, noting that the blended image inherently contains the 2D fluoroscopic image information. Depending on a user's preferences, the degree of relative brightness between the blended image and the 2D fluoroscopic image being controllable, so that one or the other can be made perceptually dominant. In another option, the 2D fluoroscopic image may be displayed alongside the blended image. An extracted catheter may be superimposed on either or both images.

The resulting 2.5-D navigation system in accordance with the present invention provides a number of benefits for RF ablation procedures, including the following.

It enables the display of the ablation catheter in the context of rendered VRT image, whose special 3D effects, such as color, shading, lighting, etc., provide the physician with a more realistic picture showing the anatomy of the heart, as compared to conventional projected fluoroscopy. This allows physicians to accurately locate, map, and ablate tissue associated with causes of arrhythmia, and hence potentially increases the success rate of AFIB ablation.

Furthermore, the process of finding good working projections is facilitated by viewing the registered 3D CT/MR volume from different angles synchronized by C-arm coordinates with the corresponding physical movement of the C-Arm of the imaging apparatus, so that the C-arm will be in the correct position for the corresponding 2D fluoroscopy without the need for capturing X-rays for recalibration. This will help to reduce the radiation dose to both the patient and the physician.

Another benefit is that the enabling of 3D road mapping of a pacing catheter using the registered VRT image can replace the conventional 2D road mapping using fluoroscopy, which saves contrast injection of the patient when viewing angulations need to be changed.

Also, catheter visualization can be enhanced through both catheter extraction techniques and blending effect with the rendered VRT image from 3D data.

A further important advantage is that a normal catheter, rather than an expensive specialized catheter, can be used for AFIB ablation under the guidance of fluoroscopy and the superimposed 3D volume, thereby reducing the expense of the cardiac mapping system, which yields reduced operational cost and medical expenses

A software prototype was built on Inspace™ to demonstrate the efficacy of 2D/3D registration and fusion articulated. Inspace™ is a Syngo™—based application. (Siemens Universal Software Platform for Medical Imaging). Illustrative figures for the application of the software prototype on AFIB ablation are described below.

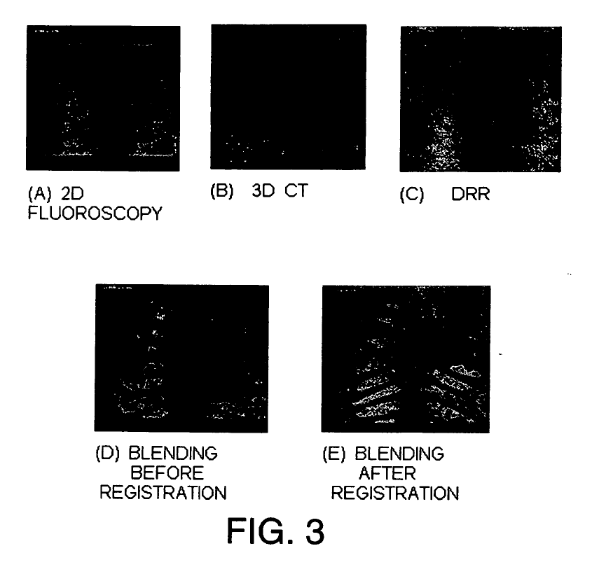

FIG. 3 shows experimental results with 2D/3D registration using an Inspace™ software prototype. (A), (B), and (C) respectively show: fluoroscopy, 3D CT, and DRR. (E) and (F) respectively show blending before registration, and blending after registration. This shows the spatial alignment of 3D CT volume and 2D fluoroscopy before and after intensity-based automatic registration. DRR is simulated for registration purposes and the rendered VRT image for CT is superimposed on fluoroscopy with the blending weight controlled by the user by way of a GUI.

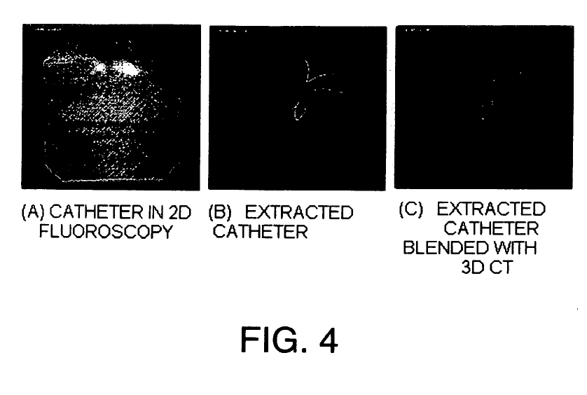

FIG. 4 shows results for catheter enhancement. (A), (B), and (C) respectively show: a catheter in 3D fluoroscopy, the extracted catheter, and the extracted catheter blended with 3D CT. The pacing catheter is displayed in the original 2D fluoroscopy, after background subtraction, edge enhancement, and automatic window-leveling, and after blending with 3D CT.

While a primary present application of the invention is in the field of AFIB ablation treatment, it is nevertheless contemplated that analogous procedures in other medical interventions or treatments, cardiac and otherwise, may benefit from the utilization and advantages of the present invention.

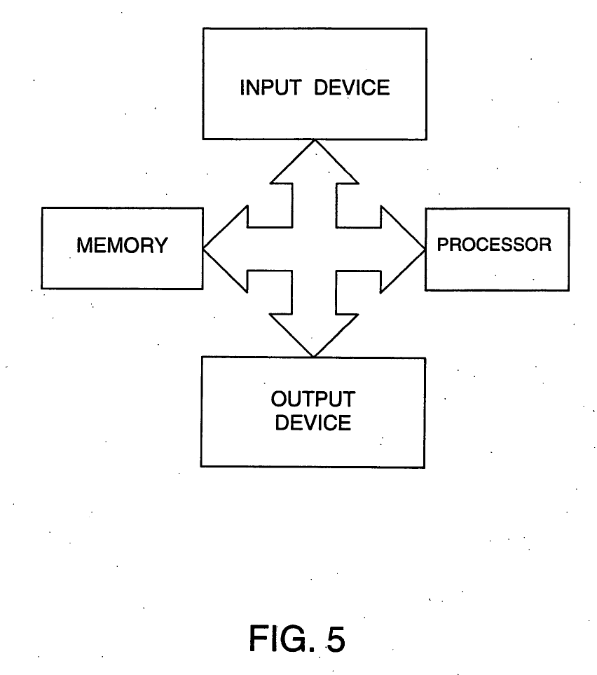

As will be apparent, the present invention is best intended to be implemented with the use and application of imaging equipment in conjunction with a programmed digital computer. FIG. 5 shows in basic schematic form a digital processor coupled for two way data communication with an input device, an output device, and a memory device for storing a program and other data. The input device is so designated in broad terms as a device for providing an appropriate image or images for processing in accordance with the present invention. For example, the input may be from an imaging device, such as a device incorporated in a CATSCAN, X-ray machine, an MRI or other device, or a stored image, or by communication with another computer or device by way of direct connection, a modulated infrared beam, radio, land line, facsimile, or satellite as, for example, by way of the World Wide Web or Internet, or any other appropriate source of such data. The output device may include a computer type display device using any suitable apparatus such as a cathode-ray kinescope tube, a plasma display, liquid crystal display, and so forth, or it may or may not include a device for rendering an image and may include a memory device or part of the memory device of FIG. 5 for storing an image for further processing, or for viewing, or evaluation, as may be convenient, or it may utilize a connection or coupling including such as are noted above in relation to the input device. The processor is operative with a program set up in accordance with the present invention for implementing steps of the invention. Such a programmed computer may interface readily through communications media such as land line, radio, the Internet, and so forth for image data acquisition and transmission. Images may be inputted directly, or by way of storage, or communication with another computer or device by way of direct connection, a modulated infrared beam, radio, land line, facsimile, or satellite as, for example, by way of the World Wide Web or Internet, or any other appropriate source of such data. The image output device may include a computer type display device using any suitable apparatus such as was referred to above, or it may include memory for storing an image for further processing, or for viewing, or evaluation, as may be convenient, or it may utilize a connection or coupling including such as are noted above in relation to the input. The processor is operative with a program set up in accordance with the present invention for implementing steps of the invention. Such a programmed computer may interface readily through communications media such as land line, radio, the Internet, and so forth for image data acquisition and transmission.

The invention may be readily implemented, at least in part, in a software memory device and packaged in that form as a software product. This can be in the form of a computer program product comprising a computer useable medium having computer program logic recorded thereon for program code for performing the method of the present invention.

The present invention has also been explained in part by way of examples using illustrative exemplary embodiments. It will be understood that the description by way of exemplary embodiments is not intended to be limiting and that, while the present invention is broadly applicable, it is helpful to also illustrate its principles, without loss of generality, by way of exemplary embodiments.

It will also be understood that various changes and substitutions not necessarily herein explicitly described may be made by one of skill in the art to which it pertains. Such changes and substitutions may be made without departing from the spirit and scope of the invention which is defined by the claims following.

What is claimed is:

1. A method for imaging for cardiac catheter guidance, comprising:
displaying a two-dimensional (2D) image of a heart;
registering said 2D image and a three-dimensional (3D) image of said heart;
deriving a blended image by fusion of said 2D image and said 3D image;
displaying said blended image;
extracting a given feature image from said 2D image; and
inserting said feature image into said 3D image.

2. A method in accordance with claim 1, wherein steps of said method are performed under automatic control.

3. A method in accordance with claim 1, wherein said step of deriving a blended image by fusion comprises blending of said 2D image and said 3D image.

4. A method in accordance with claim 3, wherein said step of blending comprises superimposing one of said 3D image and said 2D image over the other.

5. A method in accordance with claim 4, wherein relative weights of said superimposed 3D image and said 2D image are selectable by operator control.

6. A method in accordance with claim 1, wherein said registering comprises utilizing intensity-based registration.

7. A method in accordance with claim 1, wherein said registering comprises utilizing feature-based registration.

8. A method in accordance with claim 1, wherein said step of extracting a given feature image comprises extracting a catheter image.

9. A method in accordance with claim 1, comprising displaying said 3D image.

10. A method for imaging for cardiac catheter guidance, comprising:
acquiring a two-dimensional (2D) image of a heart by fluoroscopy;
acquiring a three-dimensional (3D) image of said heart by at least one of (a) computerized tomography (CT) imaging and (b) magnetic resonance (MR) imaging;
registering said 2D and said 3D images;
generating a blended image from said 2D and said 3D images;
extracting an image of a catheter from said 2D image;
displaying said blended image; and
inserting said image of said catheter into at least one of said blended image and said 3D image.

11. A method in accordance with claim 10, wherein steps of said method are performed under automatic control.

12. A method in accordance with claim 10, comprising optionally displaying said 3D image.

13. A method in accordance with claim 10, wherein said step of generating said blended image comprises:
superimposing said 2D image on top of said 3D image.

14. A method in accordance with claim 10, wherein relative intensities of said 2D image and said 3D image are controllable by a user.

15. A method in accordance with claim 10, wherein said step of displaying said blended image and said 3D image comprises:
juxtaposing said 3D image and said blended image.

16. A method in accordance with claim 10, including a step of:
superimposing a further image on said blended image.

17. A method in accordance with claim 10, wherein said step of registering comprises utilizing intensity-based registration.

18. A method in accordance with claim 17, wherein said step of utilizing intensity-based registration comprises:
digitally reconstructing a radiography (DRR) image from volumetric data from said 3D image; and
comparing quantitatively said DRR image with said 2D image to derive a rigid transformation relating an isocenter coordinate of said fluoroscopy to that of said 3D image.

19. A method in accordance with claim 18, wherein said step of utilizing intensity-based registration comprises:
utilizing 2D images from a plurality of views for intensity-based registration.

20. A method in accordance with claim 18, wherein said step of utilizing intensity-based registration comprises:
utilizing 2D images from a plurality of views for intensity-based registration so as to increase registration accuracy.

21. A method in accordance with claim 18, wherein said step of utilizing intensity-based registration comprises:
utilizing 2D images from a plurality of views for intensity-based registration so as to increase registration accuracy with respect to depth estimation.

22. A method in accordance with claim 19, wherein steps are performed under automatic control.

23. A method in accordance with claim 17, wherein said step of utilizing intensity-based registration comprises:
injecting contrast agent to highlight vessels to improve registration.

24. A method in accordance with claim 10, wherein said step of registering comprises utilizing feature-based registration.

25. A method in accordance with claim 24, comprising:
said step of acquiring a 2D image of a heart by fluoroscopy comprises utilizing a C-arm mounting for said fluoroscopy;
selecting landmarks corresponding to respective physical features present in a plurality of 2D images captured from different views corresponding respectively to different respective parameter settings of said C-arm;
computing true 3D positions of said physical features by using said parameter settings;
identifying landmark points corresponding in volumetric data of said 3D image; and
aligning said true 3D positions with corresponding respective landmark points for achieving registration.

26. A method in accordance with claim 25, wherein said step of selecting landmarks comprises utilizing said parameter settings including any of angulations, zooming effects, and similar parameter changes.

27. A method in accordance with claim 25, wherein said landmark points comprise at least 3 pairs of points.

28. A method in accordance with claim 25, wherein said landmark points comprise at least one pair of points.

29. A method in accordance with claim 25, comprising:
registering and fusing said 2D and said 3D images; and
generating a blended image from said 2D and said 3D images.

30. A method in accordance with claim 10, comprising:
adding a color component to said 3D image.

31. A method in accordance with claim 10, comprising:
extracting and highlighting edges in said 3D image.

32. A method in accordance with claim 10, comprising:
highlighting said catheter image shown in said 2D image by any of background suppression, edge enhancement filtering, and automatic window-leveling.

33. A method for imaging for cardiac catheter guidance, comprising:
acquiring a two-dimensional (2D) image of a heart by fluoroscopy including a catheter image;
acquiring a dataset for a three-dimensional (3D) image of a heart;
registering said 2D and said 3D images;
generating a blended image from said 2D and said 3D images after said registering;
extracting an image of said catheter;
displaying said blended image and said 3D image; and p1 inserting said catheter image into at least one of said blended image and said 3D image.

34. A method as recited in claim 33 including displaying said 3D image.

35. A method for imaging for cardiac catheter guidance, comprising:
displaying a two-dimensional (2D) image of a heart, including a catheter image;
extracting an image of said catheter;
deriving a three-dimensional (3D) image of said heart;
registering said 2D and 3D images;
blending said 2D and 3D images to derive a blended image;
displaying said blended image; and
inserting said catheter image into said blended image.

36. A method in accordance with claim 35, comprising displaying said 3D image.

37. A method in accordance with claim 35, wherein said step of registering comprises utilizing intensity-based registration.

38. A method in accordance with claim 37, wherein said step of utilizing intensity-based registration comprises:
digitally reconstructing a radiography (DRR) image from volumetric data from said 3D image; and
comparing quantitatively said DRR image with said 2D image to derive a rigid transformation relating an isocenter coordinate of said fluoroscopy to that of said 3D image.

39. A method in accordance with claim 38, wherein said step of utilizing intensity-based registration comprises:
utilizing 2D images from a plurality of views for intensity-based registration so as to increase registration accuracy.

40. A method in accordance with claim 38, wherein said step of utilizing intensity-based registration comprises:
utilizing 2D images from a plurality of views for intensity-based registration so as to increase registration accuracy with respect to depth estimation.

41. A method in accordance with claim 40, wherein steps are performed under automatic control.

42. A method in accordance with claim 37, wherein steps of said method are performed under automatic control.

43. A method in accordance with claim 35, wherein said step of displaying comprises:
superimposing a further image on said blended image.

44. A method in accordance with claim 39, wherein relative intensities of said blended image and said 3D image are controllable by a user.

45. A method in accordance with claim 35, wherein said step of displaying comprises:
juxtaposing said 3D image and said blended image.

46. A method in accordance with claim 41, wherein relative intensities of said blended image and said 3D image are controllable by a user.

47. A method in accordance with claim 35, wherein said step of registering comprises utilizing feature-based registration.

48. A method in accordance with claim 47, wherein said step of utilizing feature-based registration wherein:
said step of acquiring a two-dimensional (2D) image of a heart by fluoroscopy comprises utilizing a C-arm mounting for said fluoroscopy;
selecting landmarks corresponding to respective physical features present in a plurality of 2D images captured from different views corresponding respectively to different respective parameter settings of said C-arm;
computing true 3D positions of said physical features by using said parameter settings;
identifying landmark points corresponding in volumetric data of said 3D image; and
aligning said true 3D positions with corresponding respective landmark points for achieving registration.

49. A method in accordance with claim 48, wherein said step of selecting landmarks comprises utilizing said parameter settings including any of angulations, zooming effects, and similar parameter changes.

50. A method in accordance with claim 48, wherein said landmark points comprise at least 3 pairs of points.

51. A method in accordance with claim 48, wherein said landmark points comprise at least one pair of points.

52. A method in accordance with claim 35, comprising:
adding a color component to said 3D image.

53. A method in accordance with claim 35, comprising:
extracting and highlighting edges in said 3D image.

54. A method in accordance with claim 35, comprising:
highlighting said catheter shown in said 2D image by any of background suppression, edge enhancement filtering, and automatic window-leveling.

55. A system for imaging for cardiac catheter guidance, comprising:
a memory device for storing a program and other data; and
a processor in communication with said memory device, said processor being operative with said program to perform:
acquiring a two-dimensional (2D) image of a heart by fluoroscopy including a catheter;
acquiring a three-dimensional (3D) image of said heart by at least one of (a) computerized tomography (CT) imaging and (b) magnetic resonance (MR) imaging;
registering said 2D and said 3D images;
generating a blended image from said 2D and said 3D images after said registering;
extracting an image of said catheter;
displaying said blended image; and
inserting said image of said catheter into at least one of said blended image and said 3D image.

56. A system in accordance with claim 55, comprising:
displaying said 3D image.

57. A system in accordance with claim 55, wherein
said steps are performed under automatic control.

58. A system in accordance with claim 55, wherein said processor is operative with said program to perform:
displaying a further image superimposed on top of said 3D image.

59. A system in accordance with claim 55, wherein said processor is operative with said program to perform:
displaying said blended image juxtaposed with said 3D image.

60. A system in accordance with claim 55, wherein said processor is operative with said program to perform:
said registering by utilizing intensity-based registration.

61. A system in accordance with claim 60, wherein said processor is operative with said program to perform:
said step of utilizing intensity-based registration steps comprising:
digitally reconstructing a radiography (DRR) image from volumetric data from said 3D image; and
comparing quantitatively said DRR image with said 2D image to derive a rigid transformation relating an isocenter coordinate of said fluoroscopy to that of said 3D image.

62. A computer program product comprising a computer useable medium having computer program logic recorded thereon for program code for performing imaging for cardiac catheter guidance, by:
acquiring a two-dimensional (2D) image of a heart by fluoroscopy including a catheter;
acquiring a three-dimensional (3D) image of said heart by at least one of (a) computerized tomography (CT) imaging and (b) magnetic resonance (MR) imaging;
registering said 2D and said 3D images;
generating a blended image from said 2D and said 3D images;
extracting an image of said catheter;
displaying said blended image; and
inserting said image of said catheter into at least one of said blended image and said 3D image.

63. A computer program product as recited in claim 62, comprising:
displaying said 3D image.

64. A computer program product as recited in claim 62, wherein
said steps are performed under automatic control.

65. A computer program product in accordance with claim 63, including:
displaying said blended image superimposed on top of said 3D image.

66. A system in accordance with claim 63, including:
displaying said blended image juxtaposed with said 3D image.

67. A system in accordance with claim 62, wherein said processor is operative with said program to perform:
said registering by utilizing intensity-based registration.

68. A system in accordance with claim 67, wherein said processor is operative with said program to perform:
said step of utilizing intensity-based registration steps comprising:
digitally reconstructing a radiography (DRR) image from volumetric data from said 3D image; and
comparing quantitatively said DRR image with said 2D image to derive a rigid transformation relating an isocenter coordinate of said fluoroscopy to that of said 3D image.

69. A system for imaging for cardiac catheter guidance, comprising:
memory means for storing a program and other data; and
processor means in communication with said memory means, said processor means being operative with said program to perform:
acquiring a two-dimensional (2D) image of a heart by fluoroscopy including a catheter;
acquiring a three-dimensional (3D) image of said heart by at least one of (a) computerized tomography (CT) imaging and (b) magnetic resonance (MR) imaging;
registering said 2D and said 3D images;
generating a blended image from said 2D image and said 3D image;
extracting an image of said catheter;
displaying said blended image and, optionally, said 3D image; and
inserting said image of said catheter into at least one of said blended image and said 3D image.

70. A system in accordance with claim 69, wherein said processor means is operative to display said 3D image.
